(19) United States
(12) Patent Application Publication
(10) Pub. No.: US 2023/0225593 A1
(43) Pub. Date: Jul. 20, 2023

(54) IMAGE PICKUP APPARATUS, ENDOSCOPE, AND MANUFACTURING METHOD FOR IMAGE PICKUP APPARATUS

(71) Applicant: OLYMPUS CORPORATION, Tokyo (JP)

(72) Inventor: Takatoshi IGARASHI, Kamiina-gun (JP)

(73) Assignee: OLYMPUS CORPORATION, Tokyo (JP)

(21) Appl. No.: 18/125,902

(22) Filed: Mar. 24, 2023

Related U.S. Application Data
(63) Continuation of application No. PCT/JP2021/009833, filed on Mar. 11, 2021.

(51) Int. Cl. A61B 1/00; A61B 1/06

(52) U.S. Cl. CPC A61B 1/00163; A61B 1/06; A61B 1/00002

(57) ABSTRACT

An image pickup apparatus of the present invention includes a first member including an illumination optical element and an observation optical element, an image pickup device, and a second member including a signal wire and an illumination member. The first member includes a first layer substrate including an illumination window and an observation window and a second layer substrate including a first illumination element and an observation lens, the second member includes a third layer substrate including a second illumination element and an opening in which the image pickup device is disposed and a fourth layer substrate including a third illumination element and a signal wire, the first member and the second member are stacked in order of the first layer substrate, the second layer substrate, the third layer substrate, and the fourth layer substrate and integrally bonded, and the first illumination element is a light emitting element.

### CROSS REFERENCE TO RELATED APPLICATION

This application is a continuation application of PCT/JP2021/009833 filed on Mar. 11, 2021, the entire contents of which are incorporated herein by this reference.

### BACKGROUND OF THE INVENTION

### 1. Field of the Invention

The present invention relates to an image pickup apparatus provided at a distal end portion of an ultrafine-diameter endoscope, an endoscope including the image pickup apparatus, and a manufacturing method for the image pickup apparatus.

### 2. Description of the Related Art

Endoscopes including insertion sections having an elongated tube shape have conventionally been widely used in, for example, a medical field and an industrial field. Among the endoscopes, a medical endoscope used in the medical field has a function of inserting an insertion section including an observation unit into, for example, a body cavity of a living body and observing an inside of an organ or the like. The medical endoscope is configured to be able to apply various treatments to a target organ or the like by using predetermined treatment instruments as necessary. An industrial endoscope used in the industrial field is configured to be able to insert an insertion section including an observation unit into an inside of an apparatus, machine equipment, or the like such as a jet engine or a factory pipe and observe and inspect a state of flaws, corrosion, or the like in the apparatus or the machine equipment.

In general, the endoscope of this type is configured by disposing various component units on an inside of a distal end portion of an insertion section. Here, as the component units disposed on the inside of the distal end portion of the endoscope having the general configuration, there are, for example, besides an illumination unit (a light guide fiber cable or the like) for illuminating a subject and an observation unit including an image pickup device (an image sensor) and an optical lens for picking up an image of the subject, (a part on a distal end side of) a treatment instrument insertion channel for inserting a treatment instrument through an inside of the insertion section from an operation section side and inserting the treatment instrument to the distal end portion, and the like.

These component units (the illumination unit, the observation unit, the treatment instrument insertion channel, and the like) are incorporated in and bonded and fixed to respective predetermined parts in a base frame member that is provided at the distal end portion of the endoscope and in which a plurality of holes are formed by machine working or the like.

In general, the respective component units are configured by combining a plurality of components. For example, the illumination unit is configured by an optical element for illumination (a window for illumination), a light guide fiber cable for radiating illumination light, and the like. The observation unit is configured by an optical element for observation (a window for observation), an observation lens that forms an optical image of a subject, an image pickup device that photoelectrically converts the optical image formed by the observation lens and generates image data, a frame member that holds the observation lens and the image pickup device in a state in which an optical axis of the observation lens and a center position of a light receiving surface of the image pickup device are matched, a signal wire for transmitting an output signal from the image pickup device, and the like.

As the endoscope having such a form, endoscopes having various forms have been proposed by, for example, Japanese Patent No. 5639313 and generally put to practical use.

The endoscope of the related art having such a form is manufactured through a process for respectively individually manufacturing the respective component units (the illumination unit, the observation unit, the treatment instrument insertion channel, and the like) and, thereafter, incorporating the respective component units in the base frame member for each of the respective component units and bonding and fixing the respective component units.

Incidentally, in recent years, in particular, in the medical endoscope, for example, for low invasiveness, there have been strong demands for a reduction in size and a reduction in diameter of the distal end portion and the insertion section of the endoscope. Therefore, as means for realizing the reduction in size and the reduction in diameter of the endoscope, for example, downsizing of the respective components and the respective component units configuring the endoscope has been advanced.

### SUMMARY OF THE INVENTION

An image pickup apparatus according to an aspect of the present invention includes: a first member including an illumination optical element and an observation optical element respectively formed by members that transmit light; an image pickup device; and a second member including a signal wire electrically connected to the image pickup device and an illumination member disposed in a position corresponding to the illumination optical element, wherein the first member includes a first layer substrate including an illumination window and an observation window respectively formed by members that transmit light and a second layer substrate including a first illumination element and an observation lens, the second member includes a third layer substrate including a second illumination element and an opening in which the image pickup device is disposed and a fourth layer substrate including a third illumination element and a signal wire electrically connected to the image pickup device, the first member and the second member are configured in a form in which the first layer substrate, the second layer substrate, the third layer substrate, and the fourth layer substrate are stacked in this order and integrally bonded, and the first illumination element is a light emitting element.

An image pickup apparatus according to a second embodiment of the present invention includes: a first member including an illumination optical element and an observation optical element respectively formed by members that transmit light; an image pickup device; and a second member including a signal wire electrically connected to the image pickup device and an illumination member disposed in a position corresponding to the illumination optical element, wherein the first member includes a first layer substrate including an illumination window and an observation window respectively formed by members that transmit light and a second layer substrate including a first illumination element and an observation lens, the second member includes a third layer substrate including a second illumination element and an opening in which the image pickup device is disposed and a fourth layer substrate including a third illumination element and a signal wire electrically connected to the image pickup device, the first member and the second member are configured in a form in which the first layer substrate, the second layer substrate, the third layer substrate, and the fourth layer substrate are stacked in this order and integrally bonded, the first illumination element is a first opening piercing through a part of the first member in a stacking direction of the first member, and the second illumination element is a light emitting element.

An image pickup apparatus according to a third aspect of the present invention includes: a first member including an illumination optical element and an observation optical element respectively formed by members that transmit light; an image pickup device; and a second member including a signal wire electrically connected to the image pickup device and an illumination member disposed in a position corresponding to the illumination optical element, wherein the first member includes a first layer substrate including an illumination window and an observation window respectively formed by members that transmit light and a second layer substrate including a first illumination element and an observation lens, the second member includes a third layer substrate including a second illumination element and an opening in which the image pickup device is disposed and a fourth layer substrate including a third illumination element and a signal wire electrically connected to the image pickup device, the first member and the second member are configured in a form in which the first layer substrate, the second layer substrate, the third layer substrate, and the fourth layer substrate are stacked in this order and integrally bonded, each of the first layer substrate, the second layer substrate, the third layer substrate, and the fourth layer substrate includes two openings piercing through the layer substrate in a stacking direction of the layer substrate, and, when the first layer substrate, the second layer substrate, the third layer substrate, and the fourth layer substrate are stacked, the respective openings communicate to form two through-holes.

An endoscope according to an aspect of the present invention includes an image pickup apparatus including: a first member including an illumination optical element and an observation optical element respectively formed by members that transmit light; an image pickup device; and a second member including a signal wire electrically connected to the image pickup device and an illumination member disposed in a position corresponding to the illumination optical element, wherein the first member includes a first layer substrate including an illumination window and an observation window respectively formed by members that transmit light and a second layer substrate including a first illumination element and an observation lens, the second member includes a third layer substrate including a second illumination element and an opening in which the image pickup device is disposed and a fourth layer substrate including a third illumination element and a signal wire electrically connected to the image pickup device, the first member and the second member are configured in a form in which the first layer substrate, the second layer substrate, the third layer substrate, and the fourth layer substrate are stacked in this order and integrally bonded, and the first illumination element is a light emitting element.

A manufacturing method for an image pickup apparatus according to an aspect of the present invention includes manufacturing a first layer substrate including an illumination window and an observation window respectively formed by members that transmit light and an insertion opening for a channel pipe; manufacturing a second layer substrate including a first illumination element, an observation lens, and an insertion opening for the channel pipe; manufacturing a fourth layer substrate including a third illumination element, a signal wire, and an insertion opening for the channel pipe; connecting an image pickup device to the signal wire and mounting the image pickup device on the fourth layer substrate; manufacturing, on the fourth layer substrate, a third layer substrate including a second illumination element, an opening in which the image pickup device is disposed, and an insertion opening for the channel pipe; and stacking the first layer substrate, the second layer substrate, the third layer substrate, and the fourth layer substrate in order and bonding the respective layer substrates in a state in which the respective insertion openings communicate and a through-hole is formed.

### BRIEF DESCRIPTION OF THE DRAWINGS

FIG. 1 is a schematic diagram showing an overview of an entire configuration of an endoscope in an embodiment of the present invention;

FIG. 2 is a perspective view showing in a simplified manner an exterior of an image pickup apparatus (an image pickup module) in the embodiment of the present invention;

FIG. 3 is a sectional view of a cross section taken along a surface indicated by an arrow sign [3] in FIG. 2, the cross section being viewed in the arrow [3] direction;

FIG. 4 is a sectional view of a cross section taken along a surface indicated by an arrow sign [4] in FIG. 2, the cross section being viewed in the arrow [4] direction;

FIG. 5 is an exploded perspective view exploding and showing the image pickup apparatus (the image pickup module) in FIG. 2 for each of respective layer substrates;

FIG. 6 is three-view drawings respectively showing respective layers configuring a first member in the image pickup apparatus (the image pickup module) in FIG. 2;

FIG. 7 is three-view drawings respectively showing respective layers configuring a second member in the image pickup apparatus (the image pickup module) in FIG. 2;

FIG. 8 is a diagram showing a manufacturing method for the image pickup apparatus (the image pickup module) in the embodiment of the present invention and is a conceptual diagram showing a process for manufacturing respective layers of the image pickup apparatus;

FIG. 9 is a conceptual diagram showing a process for manufacturing the image pickup apparatus (the image pickup module) in the embodiment from respective layer substrates generated in FIG. 8;

FIG. 10 is a conceptual diagram showing a state in which a plurality of image pickup apparatuses (image pickup modules) in the embodiment of the present invention are generated side by side on a wafer;

FIG. 11 is a sectional view showing a first modification of the image pickup apparatus (the image pickup module) in the embodiment of the present invention;

FIG. 12 is a sectional view showing a first configuration example of a second modification of the image pickup apparatus (the image pickup module) in the embodiment of the present invention;

FIG. 13 is a sectional view showing a second configuration example of the second modification of the image pickup apparatus (the image pickup module) in the embodiment of the present invention; and

FIG. 14 is a sectional view showing a third configuration example of the second modification of the image pickup apparatus (the image pickup module) in the embodiment of the present invention.

### DETAILED DESCRIPTION OF THE PREFERRED EMBODIMENTS

In general, in the endoscope having the form of the related art disclosed by Japanese Patent No. 5639313 described above, as explained above, the base frame member and the respective component units are respectively separately configured. In the case of such a configuration, in order to surely and accurately incorporate a predetermined component unit in a predetermined part of the base frame member, for example, it is necessary to further improve machining accuracy for each of the respective components and the respective component units.

As explained above, in the endoscope having the form of the related art, since there are many assembly processes and, moreover, high assembly accuracy is always required in the respective processes, manufacturing cost tends to be large. In particular, difficulty further increases in order to assemble the components and the component units formed small for a reduction in diameter.

Further, in the configuration of the endoscope having the form of the related art, in order to realize assembly surely and at high accuracy, it is necessary to, while considering machining tolerance of each of the base frame member and the respective component units, provide predetermined clearance between a hole diameter on the base frame member side and an outer diameter on a side of the component unit inserted into the hole. Presence of such clearance is a factor of hindering a reduction in size and a reduction in diameter of the endoscope.

Completely the same problem as such a problem occurs in the respective component units provided on the inside of the distal end portion of the endoscope and configured by assembling a plurality of components. For example, in the observation unit, in particular, position accuracy of the observation lens and the image pickup device greatly affects quality of an image acquired by the image pickup unit such as resolution of the image. Because of this as well, high accuracy is always required for assembly accuracy of the image pickup unit itself.

Further, in the endoscope having the configuration of the related art, for example, in order to secure strength of the base frame member, a restriction that a predetermined minimum thickness has to be secured occurs. Therefore, for example, when a plurality of holes for disposing the respective component units are formed on the base frame member, there is a problem that there is a limit in forming the respective holes at small distances from one another.

Therefore, there is a limit in further reduction in diameter to configure, with the technique of the related art, components such as the image pickup unit incorporated in the endoscope of the related art of this type.

Accordingly, according to the present invention, it is possible to provide an image pickup apparatus including a configuration that can simplify a manufacturing process and contribute to a reduction in manufacturing cost while realizing a reduction in size and an extreme reduction in diameter, an endoscope including the image pickup apparatus, and a manufacturing method for the image pickup apparatus.

The present invention is explained below with reference to embodiments shown in the figures. Respective drawings used for the following explanation are schematically shown. In order to show respective constituent elements in recognizable degrees of sizes on the drawings, dimensional relations, scales, and the like of respective members are sometimes shown differently for each of the constituent elements. Therefore, the present invention is not limited to only forms shown in the figures concerning quantities of the respective constituent elements, shapes of the respective constituent elements, ratios of sizes of the respective constituent elements, relative positional relations of the respective constituent elements, and the like described in the respective drawings.

### Embodiment

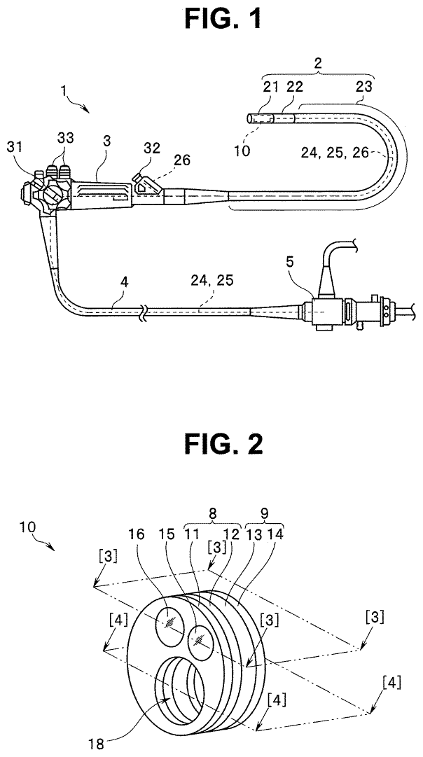

First, before a detailed configuration of an image pickup apparatus in an embodiment of the present invention is explained, an overview of an entire configuration of an endoscope in the present embodiment to which the image pickup apparatus is applied is explained with reference to FIG. 1. FIG. 1 is a schematic diagram showing the overview of the entire configuration of the endoscope in the embodiment of the present invention.

As shown in FIG. 1, an endoscope 1 in the present embodiment includes an endoscope insertion section (hereinafter simply abbreviated as insertion section) 2 formed in an elongated shape and inserted into a subject, an operation section 3 consecutively connected to a proximal end side of the insertion section 2 and grasped by a surgeon (a using person or a user of the endoscope 1), a flexible universal cord 4 extending from the operation section 3, and a connector 5 provided at a proximal end portion of the universal cord 4 and for connecting the endoscope 1 to external peripheral equipment (a not-shown control unit and the like).

The insertion section 2 is configured by an elongated tubular member having flexibility. The insertion section 2 is configured by consecutively connecting a distal end portion 21, a bending section 22, and a flexible tube section 23 in order from a distal end side along a longitudinal axis (hereinafter referred to as major axis).

The distal end portion 21 is provided at a distalmost portion of the insertion section 2. On an inside of the distal end portion 21, for example, an image pickup module 10, which is a distal end rigid member and is the image pickup apparatus in the present embodiment is disposed. The image pickup module 10 includes, for example, an observation unit (an observation apparatus) that observes an examination target site in the subject and picks up an image and an illumination unit (an illumination apparatus) that radiates illumination light toward a predetermined region including the examination target site. Note that a detailed configuration of the image pickup module 10, which is the image pickup apparatus of the present embodiment, is explained below.

The bending section 22 is provided on the proximal end side of the distal end portion 21 and includes a mechanism for bendably configuring a part within a predetermined range on the distal end side of the insertion section 2. The bending section 22 is configured by, for example, a plurality of bending pieces coupled along the major axis of the insertion section 2. The bending section 22 is actively operated to bend using a well-known bending mechanism (not shown). Detailed explanation of a configuration of the bending section 22 is omitted assuming that the configuration is well-known and the bending section 22 has the same configuration as a bending section applied to an endoscope of the same type in the related art.

The flexible tube section 23 is provided to extend from a proximal end side of the bending section 22 and configured by a tubular member having flexibility that is passively bendable.

Note that, besides a plurality of bending wires (not shown) included in the bending mechanism of the bending section 22 and a signal cable 25 extended from the observation unit of the distal end portion 21, an illumination member 24 such as a light guide fiber cable that supplies illumination light to the illumination unit of the distal end portion 21, a treatment instrument insertion channel 26, and the like are inserted through an inside of the insertion section 2.

The operation section 3 is formed by a grasping section that is a part grasped by fingers or the like of the surgeon (the using person or the user), a main body section including the grasping section and formed to be capable of housing various structures on an inside, and the like. A proximal end portion of the flexible tube section 23 of the insertion section 2 is consecutively connected to an end portion of the operation section 3. In other words, an end portion of the insertion section 2 is guided to an inside of the operation section 3 and fixed on the inside of the operation section 3 using predetermined well-known means.

On an outer surface of the operation section 3, a bending operation member 31 for performing bending operation of the bending section 22, an operation switch 33 for performing various kinds of operation of not-shown peripheral equipment (an information processing apparatus, a light source apparatus, an air feeding and water feeding apparatus, a suction apparatus, and the like), and the like are disposed.

A treatment instrument insertion section 32 is formed in a predetermined part closer to a distal end of the operation section 3. The treatment instrument insertion section 32 is formed to include a treatment instrument insertion opening (not shown) that communicates with the treatment instrument insertion channel 26. The treatment instrument insertion opening is an opening into which various treatment instruments (for example, biological forceps and a laser knife) are inserted.

In other words, by inserting a predetermined treatment instrument from the treatment instrument insertion opening into the treatment instrument insertion channel 26, the treatment instrument is configured to project from a channel insertion hole 18 at a distal end portion to a front of the distal end portion and to be able to be inserted into the subject.

The universal cord 4 extends from a side surface of the operation section 3. The connector 5 and the like are consecutively connected to a distal end of the universal cord 4. The universal cord 4 causes the illumination member 24 such as the light guide cable, the signal cable 25, and the like to communicate in an inside. The universal cord 4 is mechanically and electrically connected to the external peripheral equipment (not shown) to the endoscope 1 through the connector 5.

In the endoscope 1 in the present embodiment configured as explained above, a configuration of the image pickup module 10, which is the distal end rigid member provided on the inside of the distal end portion 21 and is the image pickup apparatus in the present embodiment, is explained below with reference to FIG. 2 to FIG. 7.

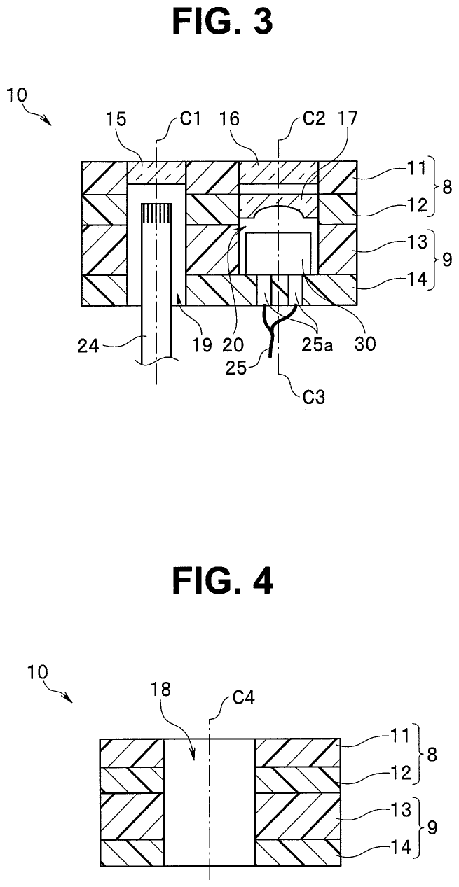

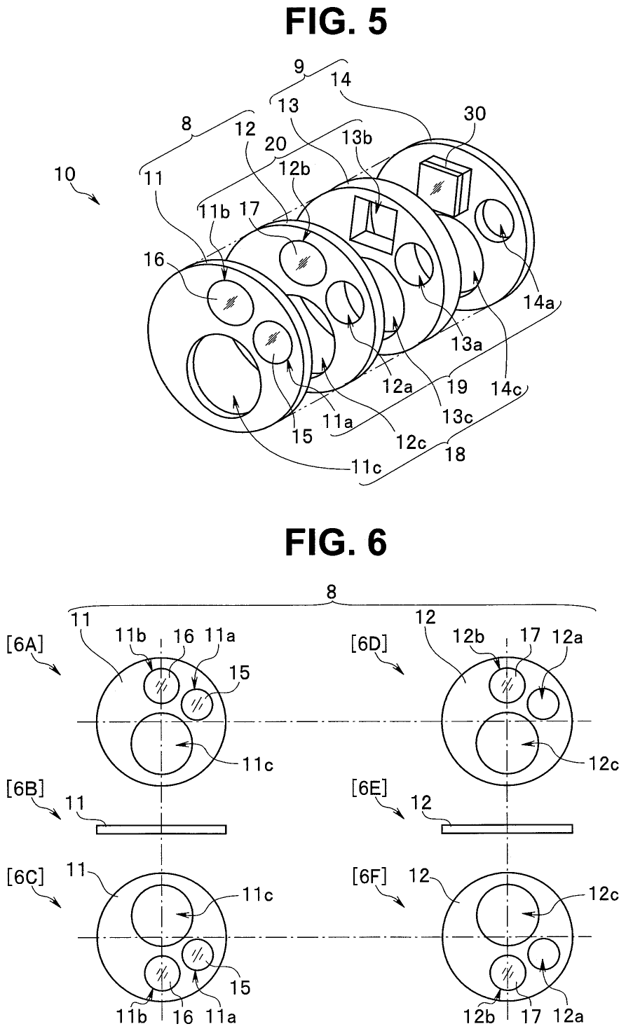

FIG. 2 is a perspective view showing in a simplified manner an exterior of the image pickup apparatus (the image pickup module) in the present embodiment. FIG. 3 is a sectional view of a cross section taken along a surface indicated by an arrow sign [3] in FIG. 2, the cross section being viewed in the arrow [3] direction. FIG. 4 is a sectional view of a cross section taken along a surface indicated by an arrow sign [4] in FIG. 2, the cross section being viewed in the arrow [4] direction. FIG. 5 is an exploded perspective view exploding and showing the image pickup apparatus (the image pickup module) in FIG. 2 for each of respective layer substrates.

FIG. 6 is three-view drawings respectively showing respective layers configuring a first member in the image pickup apparatus (the image pickup module) in FIG. 2. Note that, in FIG. 6, a sign [6A] indicates a front view of a first layer substrate, a sign [6B] indicates a side view of the first layer substrate, and a sign [6C] indicates a rear view of the first layer substrate. In FIG. 6, a sign [6D] indicates a front view of a second layer substrate, a sign [6E] indicates a side view of the second layer substrate, and a sign [6F] indicates a rear view of the second layer substrate.

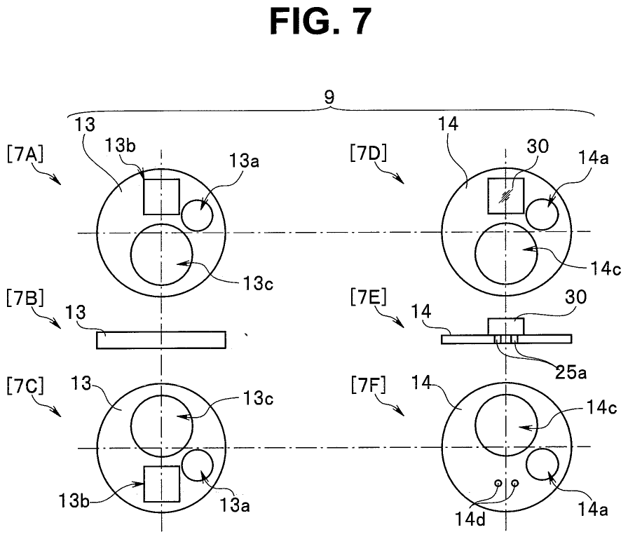

FIG. 7 is three-view drawings respectively showing respective layers configuring a second member in the image pickup apparatus (the image pickup module) in FIG. 2. Note that, in FIG. 7, a sign [7A] indicates a front view of a third layer substrate, a sign [7B] indicates a side view of the third layer substrate, and a sign [7C] indicates a rear view of the third layer substrate. In FIG. 7, a sign [7D] indicates a front view of a fourth layer substrate, a sign [7E] indicates a side view of the fourth layer substrate, and a sign [7F] indicates a rear view of the fourth layer substrate.

The image pickup module 10 is a distal end rigid member having a form formed in a substantially columnar shape as a whole as shown in FIG. 2. As explained in detail below, on an inside of the image pickup module 10, component units such as observation units (16, 17, and 30) and illumination units (15 and 24) are integrally housed and the channel insertion hole 18 through which a distal end portion of a channel pipe (not shown) of the treatment instrument insertion channel 26 is inserted is formed. An outer surface of the image pickup module 10 is covered by a cover member (not shown in FIG. 1) having insulation properties.

The image pickup module 10 is configured by, as shown in FIG. 2 to FIG. 4, a first member 8, a second member 9, and an image pickup device 30 (not shown in FIG. 2; explained in detail below). The first member 8 and the second member 9 are an integrated component member integrally bonded in a stacked form. The image pickup device 30 is disposed on an inside of the integrated component member of the first member 8 and the second member 9.

The image pickup device 30 is a photoelectric conversion element that receives an optical image of the examination target site formed by an image pickup optical system (17) explained below and converts the optical image of the examination target site into an electric signal. As the image pickup device 30, for example, a CCD (charge coupled device)-type image sensor or a CMOS (complementary metal oxide semiconductor)-type image sensor is applied.

The first member 8 includes illumination optical elements (an illumination window 15 and an illumination optical path 19) and observation optical elements (an observation window 16, an observation lens 17, and an observation optical path 20). In the first member 8, the channel insertion hole 18 through which the distal end portion of the channel pipe (not shown) of the treatment instrument insertion channel 26 is inserted is formed.

The illumination window 15 among the illumination optical elements is formed by a member (for example, a transparent resin material) that transmits light. The illumination window 15 has a function of transmitting illumination light emitted from the illumination member 24 explained below. Consequently, the illumination light from the illumination member 24 illuminates the examination target site in the subject in a front of the image pickup module 10. The illumination window 15 also has a function of watertightly sealing the inside of the image pickup module 10 against an outside. The illumination optical path 19 among the illumination optical elements is a passage that is disposed to be inserted through a distal end portion of the illumination member 24 explained below and through which the illumination light from the illumination member 24 passes to the illumination window 15, is an illumination element, and is a first through-hole.

The observation window 16 among the observation optical elements is formed by a member (for example, a transparent resin material) that transmits light. The observation window 16 allows reflected light from the examination target site present in the front of the image pickup module 10 to pass and takes the reflected light into the inside of the image pickup module 10. Consequently, the reflected light from the examination target site taken into the inside of the image pickup module 10 is transmitted through the observation lens 17 explained below and, thereafter, reaches a light receiving surface of the image pickup device 30. The observation window 16 also has a function of watertightly sealing the inside of the image pickup module 10 against the outside. The observation window 16 may be configured as a part of an image pickup optical system formed by combination with the observation lens 17 explained below.

The observation lens 17 among the observation optical elements is formed by a member (for example, a transparent resin material) that transmits light. The observation lens 17 configures an image pickup optical system that transmits reflected light from the examination target site having passed through the observation window 16 and taken into the inside of the image pickup module 10 and forms an optical image of the examination target site.

The observation optical path 20 among the observation optical elements is a passage in which the image pickup device 30 explained below is disposed and through which reflected light from the examination target site made incident from the observation window 16 passes to the light receiving surface of the image pickup device 30.

Note that, in a configuration example in the present embodiment, a configuration is illustrated in which the illumination window 15, the observation window 16, and the observation lens 17 provided in the first member 8 are formed by the transparent resin material or the like that transmits light but are not limited to this form. Another material that can be formed integrally with layer substrates respectively corresponding to the illumination window 15, the observation window 16, and the observation lens 17, for example, a transparent member such as glass can also be applied as the illumination window 15, the observation window 16, and the observation lens 17.

The second member 9 includes image pickup signal wires 25a, a part of the illumination optical path 19, and the illumination member 24. In the second member 9, the channel insertion hole 18 through which the distal end portion of the channel pipe (not shown) of the treatment instrument insertion channel 26 is inserted is formed.

The image pickup signal wires 25a are wiring members electrically connected to the image pickup device 30 and for transmitting an image pickup signal outputted from the image pickup device 30, a control signal to the image pickup device 30, and the like. Note that the signal cable 25 is connected to the other end sides of the image pickup signal wires 25a (see FIG. 3). The image pickup signal wires 25a are, for example, Si through-electrodes (TSVs; through-silicon vias).

The illumination member 24 is a component member including a light emitting body that radiates illumination light for illuminating the examination target site. As the illumination member 24, a light emitting element such as a light guide fiber cable or a light emitting diode (LED) is applied.

As shown in FIG. 2 to FIG. 7, in the configuration of the image pickup module 10 illustrated in the present embodiment, an example is explained in which the first member 8 is configured in a form in which a first layer substrate 11 and a second layer substrate 12 are stacked and integrally bonded. An example is explained in which, like the first member 8 described above, the second member 9 is configured in a form in which a third layer substrate 13 and a fourth layer substrate 14 are stacked and integrally bonded. The first member 8 and the second member 9 are bonded between a rear surface side of the second layer substrate 12 and a front surface side of the third layer substrate 13 to be integrally configured.

As the first layer substrate 11, the second layer substrate 12, the third layer substrate 13, and the fourth layer substrate 14, a thin-plate circular rigid substrate member made of, for example, a silicon material (a silicon wafer), a glass material, a resin material, a metal material, or the like is applied. In the configuration example in the present embodiment, a configuration example is explained in which a rigid substrate member made of a resin material is applied as the rigid substrate member of the respective layer substrates 11 to 14.

In the first layer substrate 11 of the first member 8, as indicated by signs [6A], [6B], and [6C] in FIG. 5 and FIG. 6, an illumination opening 11a, an observation opening 11b, and a channel opening 11c are formed. The respective openings (11a, 11b, and 11c) are formed as through-holes orthogonal to a plane of the first layer substrate 11. The illumination window 15 is integrally formed in the illumination opening 11a. The observation window 16 is integrally formed in the observation opening 11b. Note that the channel opening 11c is a through-hole through which the distal end portion of the channel pipe (not shown) is inserted.

In the second layer substrate 12 of the first member 8, as indicated by signs [6D], [6E], and [6F] in FIG. 5 and FIG. 6, an illumination opening 12a, an observation opening 12b, and a channel opening 12c are formed. The respective openings (12a, 12b, and 12c) are formed as through-holes orthogonal to a plane of the second layer substrate 12. The observation lens 17 is integrally formed in the observation opening 12b. The illumination opening 12a is a through-hole through which the distal end portion of the illumination member 24 is inserted and disposed or that becomes a part of a passage (the illumination optical path 19) for allowing illumination light of the illumination member 24 to pass. The illumination opening 12a is referred to as first opening or first illumination element as well. Note that, like the channel opening 11c described above, the channel opening 12c is a through-hole through which the distal end portion of the channel pipe (not shown) is inserted.

In the third layer substrate 13 of the second member 9, as indicated by signs [7A], [7B], and [7C] in FIG. 5 and FIG. 7, an illumination opening 13a, an image pickup device disposition opening 13b, and a channel opening 13c are formed. The respective openings (13a, 13b, and 13c) are formed as through-holes orthogonal to a plane of the third layer substrate 13. Like the illumination opening 12a described above, the illumination opening 13a is a through-hole through which the distal end portion of the illumination member 24 is inserted and disposed or that becomes a part of the passage (the illumination optical path 19) for allowing illumination light of the illumination member 24 to pass. The illumination opening 13a is referred to as second opening or second illumination element as well. Like the channel openings 11c and 12c described above, the channel opening 13c is a through-hole through which the distal end portion of the channel pipe (not shown) is inserted.

The image pickup device 30 is integrally disposed in the image pickup device disposition opening 13b. The image pickup device 30 is a form of a chip size package (CSP), on a surface of which on an opposite side of the light receiving surface of the image pickup device 30 an external input/output terminal (not shown) is formed. In the configuration illustrated in the present embodiment, it is assumed that the image pickup device 30 is mounted on a surface of the fourth layer substrate 14 explained below. Therefore, a thickness dimension of the third layer substrate 13 is set such that light condensed by the image pickup optical system (the observation window 16 and the observation lens 17) is focused (forms an image) on the light receiving surface of the image pickup device 30. In other words, the thickness dimension of the third layer substrate 13 is set such that a focal position of the image pickup optical system (16, 17) and a position of the light receiving surface of the image pickup device 30 coincide. More preferably, the thickness dimension of the third layer substrate 13 is set such that an optical axis of the observation lens 17 passes a center point of the light receiving surface of the image pickup device 30.

In other words, the thickness dimension of the third layer substrate 13 is defined as appropriate by a focal length of the image pickup optical system (the observation window 16 and the observation lens 17) and a package thickness dimension (the position of the light receiving surface) of the image pickup device 30 itself.

In the fourth layer substrate 14 of the second member 9, as indicated by signs [7D], [7E], and [7F] in FIG. 5 and FIG. 7, an illumination opening 14a, a channel opening 14c, and wiring openings 14d are formed. The respective openings (14a, 14c, and 14d) are formed as through-holes orthogonal to a plane of the fourth layer substrate 14. Like the illumination openings 12a and 13a described above, the illumination opening 14a is a through-hole through which the distal end portion of the illumination member 24 is inserted and disposed or that becomes a part of the passage (the illumination optical path 19) for allowing illumination light of the illumination member 24 to pass. The illumination opening 14a is referred to as third opening or third illumination element as well. Like the channel openings 11c, 12c, and 13c described above, the channel opening 14c is a through-hole through which the distal end portion of the channel pipe (not shown) is inserted. The image pickup signal wires 25a are formed on insides of the wiring openings 14d.

As explained above, the image pickup device 30 is mounted in a predetermined position on the surface of the fourth layer substrate 14. In other words, the external input/output terminal (not shown) of the image pickup device 30 is connected to the image pickup signal wires 25a. The signal cable 25 is connected to the image pickup signal wires 25a (see FIG. 3). The image pickup signal wires 25a and the signal cable 25 may be connected using a relay board. Consequently, the image pickup signal wires 25a play a role of relaying electric connection between the image pickup device 30 and the signal cable 25 across the fourth layer substrate 14.

Note that, in the configuration example in the present embodiment, a form is illustrated in which the image pickup device 30 is mounted on the surface of the fourth layer substrate 14. However, the image pickup device 30 is not limited to this form. As a configuration example different from the present embodiment, it is also possible to adopt, as the image pickup device 30, for example, a form in which a silicon substrate is used as the rigid substrate member and an image pickup device is directly formed in the silicon substrate.

The first layer substrate 11, the second layer substrate 12, the third layer substrate 13, and the fourth layer substrate 14 configured as explained above are bonded in a form in which the first layer substrate 11, the second layer substrate 12, the third layer substrate 13, and the fourth layer substrate 14 are stacked in order. In this case, the respective layer substrates (11 to 14) are accurately positioned in a plane in a direction orthogonal to a stacking direction of the respective layer substrates (11 to 14) and stacked in order to make it possible to surely realize functions that the layer substrates (11 to 14) respectively have.

In other words, more specifically, for example, when the respective layer substrates (11 to 14) are stacked, the illumination openings 11a, 12a, 13a, and 14a are positioned in substantially coinciding positions in the stacking direction of the respective layer substrates (11 to 14). More specifically, for example, the respective layer substrates (11 to 14) are stacked such that respective center axes (see a sign C1 in FIG. 3) of the respective illumination openings (11a to 14a) respectively substantially coincide. Consequently, the respective illumination openings (11a, 12a, 13a, and 14a) form the illumination optical path 19 that is one through-hole communicating in the stacking direction. The distal end portion of the illumination member 24 is inserted into and disposed in the illumination optical path 19. Consequently, an illumination unit is formed by the illumination member 24, the illumination optical path 19, and the illumination window 15.

Similarly, when the respective layer substrates (11 to 14) are stacked, the observation openings 11b and 12b are positioned in substantially coinciding positions in the stacking direction of the respective layer substrates (11 to 14). More specifically, for example, the respective layer substrates (11 to 14) are stacked such that respective center axes (see a sign C2 in FIG. 3) of the respective observation openings (11b and 12b) respectively substantially coincide. Consequently, the respective observation openings (11b and 12b) form the observation optical path 20 that is one through-hole communicating in the stacking direction.

Further, the center axis C3 of the image pickup device disposition opening 13b and the center axis C2 of the observation optical path 20 are positioned to substantially coincide. This means that the center axis C3 of the image pickup device 30 disposed in the image pickup device disposition opening 13b and the optical axis C2 of the image pickup optical systems (16 and 17) disposed in the observation openings 11b and 12b are made to substantially coincide. Consequently, the observation window 16, the observation lens 17, and the image pickup device 30 are disposed in a form in which the observation window 16, the observation lens 17, and the image pickup device 30 are arrayed in a row in order in a direction along the optical axes C2 and C3, whereby an observation unit is formed.

Similarly, when the respective layer substrates (11 to 14) are stacked, the channel openings 11c, 12c, 13c, and 14c are positioned in substantially coinciding positions in the stacking direction of the respective layer substrates (11 to 14). More specifically, for example, the respective layer substrates (11 to 14) are stacked such that respective center axes (see a sign C4 in FIG. 4) of the respective channel openings (11c, 12c, 13c, and 14c) respectively substantially coincide. Consequently, the respective channel openings (11c, 12c, 13c, and 14c) form the channel insertion hole 18 that is one through-hole communicating in the stacking direction and is a second through-hole. The distal end portion of the channel pipe (not shown) is inserted through the channel insertion hole 18 to configure a distal end side of the treatment instrument insertion channel 26. Consequently, a treatment instrument (not shown) inserted from the treatment instrument insertion section 32 reaches the distal end portion 21 of the insertion section 2 of the endoscope 1 through the treatment instrument insertion channel 26. A distal end portion of the treatment instrument projects forward from a distal end face of the image pickup module 10 through the channel insertion hole 18 and is led out into the subject.

Note that, in the configuration example in the present embodiment, a form is illustrated in which the channel insertion hole 18 through which the distal end portion of the channel pipe (not shown) of the treatment instrument insertion channel 26 is inserted is formed and the channel insertion hole 18 is formed as a part of a component on the distal end side of the treatment instrument insertion channel 26. However, a configuration in which a treatment instrument insertion channel is made unnecessary as a constituent element of an endoscope is also conceivable. When the present invention is applied to the endoscope having such a configuration, the channel insertion hole 18 does not always need to be formed in the image pickup module 10 functioning as a distal end rigid member of a distal end portion of the endoscope. The channel insertion hole 18 can be omitted from constituent elements of the image pickup module 10.

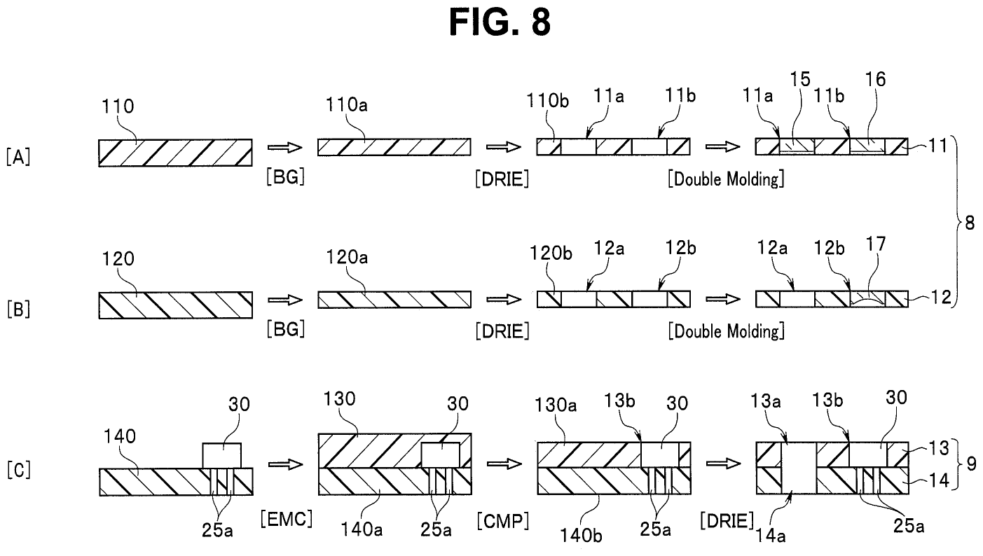

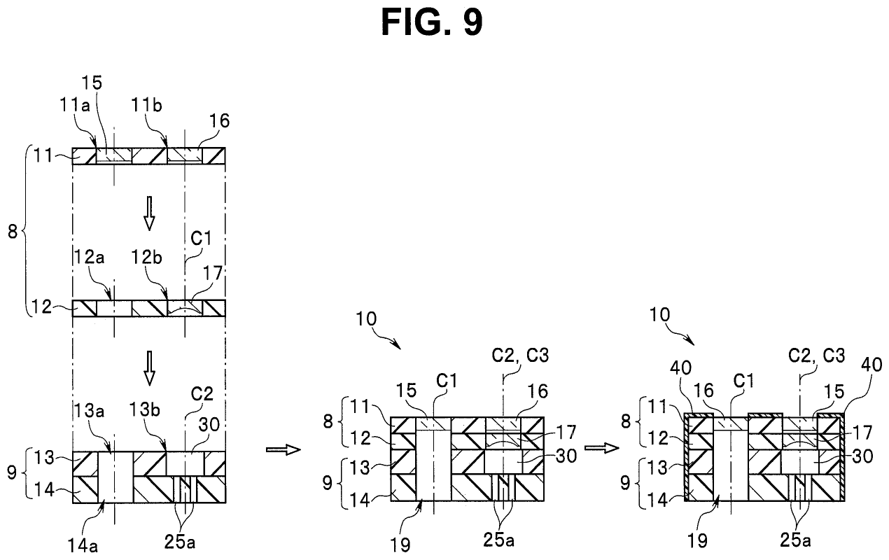

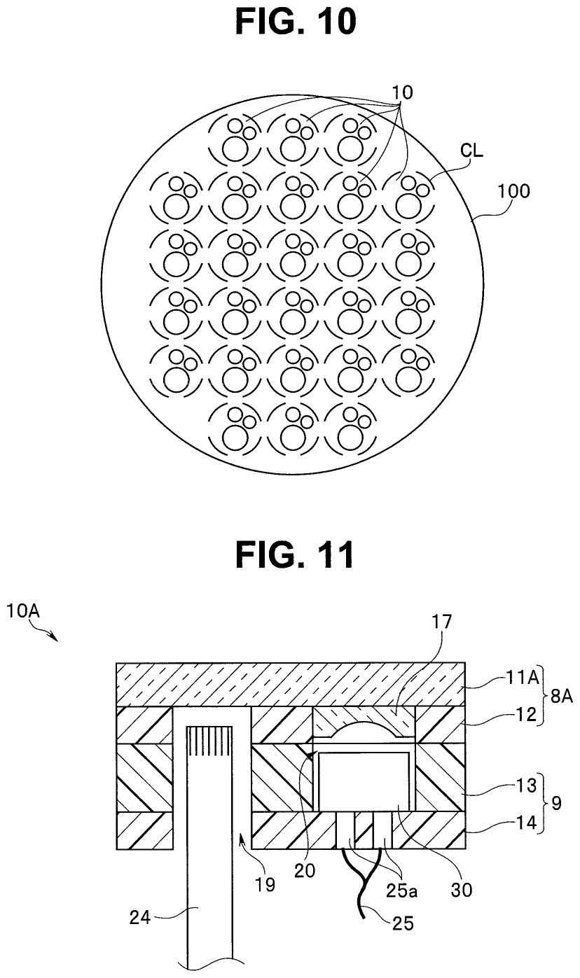

A manufacturing method for the image pickup module in the present embodiment configured as explained above is explained below with reference to FIG. 8, FIG. 9, and FIG. 10.

FIG. 8, FIG. 9, and FIG. 10 are diagrams showing a manufacturing method for the image pickup apparatus (the image pickup module) in the present embodiment. Among the figures, FIG. 8 is a conceptual diagram showing a process for manufacturing respective layers configuring the image pickup apparatus (the image pickup module) in the present embodiment. FIG. 9 is a conceptual diagram showing a process for manufacturing the image pickup apparatus (the image pickup module) in the present embodiment from respective layer substrates generated in FIG. 8. FIG. 10 is a conceptual diagram showing a state in which a plurality of image pickup apparatuses (image pickup modules) in the present embodiment are generated side by side on a wafer.

The manufacturing method for the image pickup module 10 substantially includes steps explained below:

(1) manufacturing the first layer substrate 11 including the illumination window 15 and the observation window 16 (see FIG. 8[A]);
(2) manufacturing the second layer substrate 12 including the illumination opening 12a and the observation lens 17 (see FIG. 8[B]);
(3) manufacturing the fourth layer substrate 14 including the illumination opening 14a and the image pickup signal wires 25a and mounting the image pickup device 30 on the surface of the fourth layer substrate 14 (connecting the image pickup device 30 to the image pickup signal wires 25a) (see FIG. 8[C]);
(4) manufacturing (stacking), on the fourth layer substrate, the third layer substrate 13 including the illumination opening 13a and the image pickup device disposition opening 13b (see FIG. 8[C]);
(5) bonding the first layer substrate 11, the second layer substrate 12, the third layer substrate 13, and the fourth layer substrate 14 in a form in which the first layer substrate 11, the second layer substrate 12, the third layer substrate 13, and the fourth layer substrate 14 are stacked in this order (see FIG. 9); and
(6) covering, with an insulative cover member 40, an outer surface of a component unit obtained by stacking and bonding the first layer substrate 11, the second layer substrate 12, the third layer substrate 13, and the fourth layer substrate 14.

In short, the image pickup module 10 in the present embodiment is manufactured by respectively manufacturing the first layer substrate 11, the second layer substrate 12, the third layer substrate 13, and the fourth layer substrate 14 and stacking and bonding the layer substrates.

First, as shown in FIG. 8[A], for example, back grind machining is applied to a rigid substrate member (hereinafter referred to as rigid substrate) 110 formed by, for example, silicon or resin to mold a rigid substrate 110a having a desired thickness functioning as the first layer substrate 11.

Subsequently, a rigid substrate 110b in which the illumination opening 11a, the observation opening 11b, and the channel opening 11c (not shown in FIG. 8) are formed by applying, for example, deep reactive ion etching (DRIE) to the rigid substrate 110a is molded.

Subsequently, molding by resin or the like that transmits light is performed on the illumination opening 11a and the observation opening 11b of the rigid substrate 110b. In other words, the illumination window 15 and the observation window 16 are molded by molding in the illumination opening 11a and the observation opening 11b. In this way, the first layer substrate 11 in which the illumination window 15 and the observation window 16 are integrally molded is manufactured.

Subsequently, as shown in FIG. 8[B], like the first layer substrate 11, a rigid substrate 120a having a desired thickness functioning as the second layer substrate 12 is molded by applying, for example, BG machining to a rigid substrate 120 formed by, for example, silicon or resin.

Subsequently, a rigid substrate 120b in which the illumination opening 12a, the observation opening 12b, and the channel opening 12c (not shown in FIG. 8) are formed by applying, for example, the DRIE to the rigid substrate 120a is molded.

Subsequently, molding by resin or the like that transmits light is performed on the observation opening 12b of the rigid substrate 120b to mold the observation lens 17. In this way, the second layer substrate 12 on which the observation lens 17 is integrally molded is manufactured.

The first layer substrate 11 and the second layer substrate 12 are stacked and bonded, whereby the first member 8 is manufactured.

Subsequently, the second member 9 in which the third layer substrate 13 and the fourth layer substrate 14 are bonded is manufactured. For that purpose, first, the fourth layer substrate 14 is manufactured.

In other words, first, as shown in FIG. 8[C], the image pickup signal wires 25a piercing through a rigid substrate 140 are formed in the rigid substrate 140. A CSP package of the image pickup device 30 is, for example, SMT (surface mount technology)-mounted on a surface of the rigid substrate 140. Consequently, the fourth layer substrate 14 is manufactured.

Subsequently, a resin layer 130 to be a third layer substrate is formed on the surface (a surface on a side on which the image pickup device 30 is mounted) of the rigid substrate 140. The resin layer 130 is formed to seal the image pickup device 30 by, for example, an epoxy resin sealing material (EMC: epoxy molding compound). Consequently, the resin layer 130 and the rigid substrate 140 are molded in a form in which the resin layer 130 and the rigid substrate 140 are stacked and bonded.

Subsequently, the resin layer 130 is polished by, for example, chemical mechanical planarization (CMP) to mold a resin layer 130a having a thickness of the third layer substrate 13.

Subsequently, the third layer substrate 13 and the fourth layer substrate 14 in which the illumination openings 13a and 14a, the image pickup device disposition opening 13b, and the channel openings 13c and 14c (not shown in FIG. 8) are formed by applying the DRIE or the like to a substrate having a form in which the resin layer 130a and the rigid substrate 140 are stacked and bonded are molded. At this time, the third layer substrate 13 and the fourth layer substrate 14 are manufactured as the second member 9 having a form in which the third layer substrate 13 and the fourth layer substrate 14 are stacked and bonded.

The first member 8 and the second member 9 manufactured as explained above are bonded in a stacked state as shown in FIG. 9. Consequently, the image pickup module 10 is formed. At this time, the illumination openings 11a to 14a provided in the respective layer substrates 11 to 14 are positioned to make the center axes C1 coincide such that the illumination openings 11a to 14a become the illumination optical path 19, the image pickup openings 11b to 13b are positioned to make the optical axes C2 and C3 of the observation window 16 and the observation lens 17 and the image pickup device 30 coincide such that the image pickup openings 11b to 13b become the observation optical path 20, and the channel openings 11c to 14c are positioned and stacked to make the center axes C4 (not shown in FIG. 9; see FIG. 3) coincide such that the channel openings 11c to 14c become the channel insertion hole 18.

The insulative cover member 40 is applied to, by, for example, insert molding or Parylene coat, the outer surface of the image pickup module 10 formed in this way.

The illumination member 24 such as the light guide cable is disposed in the illumination optical path 19. Consequently, an illumination unit is configured by the illumination window 15 and the illumination member 24 (not shown in FIG. 9; see FIG. 3).

A plurality of image pickup modules 10 manufactured as explained above are simultaneously formed by manufacturing a bonded wafer 100 molded by a wafer process shown in FIG. 10. The bonded wafer 100 is singulated into the plurality of image pickup modules 10 by being cut off by, for example, plasma dicing along a cutting line CL. Consequently, the individual image pickup modules 10 are manufactured as, for example, ultrafine-diameter image pickup modules having a diameter of approximately 2.5 mm. As a thickness dimension of each of the respective layer substrates, for example, a thickness dimension of approximately 0.2 to 0.4 mm is applied, whereby the image pickup module 10 is manufactured as an extremely small image pickup module having a thickness dimension of, for example, approximately 1 mm.

As explained above, according to the embodiment explained above, the image pickup module 10 is formed that includes the first member 8 including the illumination optical elements (the illumination window 15 and the illumination optical path 19) and the observation optical elements (the observation window 16, the observation lens 17, and the observation optical path 20) respectively formed by members that transmit light, the image pickup device 30, and the second member 9 including the image pickup signal wires 25a electrically connected to the image pickup device 30 and the illumination member 24 disposed in a position corresponding to the illumination optical elements, the image pickup module 10 being configured by stacking and integrally bonding the first member 8 and the second member 9.

With such a configuration, since respective component units and a frame member to be a base that fixes and holds the respective component units can be integrally formed, it is possible to make clearance required in assembly unnecessary compared with the related art of separately manufacturing the respective component units and, thereafter, assembling the respective component units to the frame member. Therefore, it is possible to contribute to a further reduction in diameter of the distal end portion in the endoscope 1 and, at the same time, contribute to a reduction in manufacturing cost.

Note that, in the configuration example in the present embodiment, a form is illustrated in which the first member 8 is configured by the two layer substrates 11 and 12 and the second member 9 is configured by the two layer substrates 13 and 14. However, the first member 8 and the second member 9 are not limited to this form. For example, each of the first member 8 and the second member 9 may be configured by one layer. One of the first member 8 and the second member 9 can be configured by one layer and the other can be formed in a multilayer structure. Further, each of the first member 8 and the second member 9 can be configured as a structure including three or more layers. In the image pickup module 10 functioning as the image pickup apparatus of the present invention, as structure of the layer substrates configuring the first member 8 and the second member 9, the layer substrates can be configured in an appropriate combination.

In the manufacturing method for the image pickup module 10 in the present embodiment explained above, the channel insertion hole 18 is formed by respectively forming the channel openings 11c, 12c, 13c, and 14c in the first layer substrate 11, the second layer substrate 12, the third layer substrate 13, and the fourth layer substrate 14 and, thereafter, stacking the respective layer substrates. However, the channel insertion hole 18 is not limited to this form. For example, the channel insertion hole 18 may be formed by stacking and bonding the first layer substrate 11, the second layer substrate 12, the third layer substrate 13, and the fourth layer substrate 14 and, thereafter, forming through-holes at a time with the DRIE or the like. The illumination optical path 19 can be formed in the same manner as explained above.

If the through-holes are formed after the respective layer substrates are stacked and bonded first in this way, the channel insertion hole 18 and the illumination optical path 19 can be formed by one process. Accordingly, a manufacturing process can be reduced. Further, compared with a manufacturing method of providing openings in respective layer substrates and, thereafter, stacking and bonding the respective layer substrates, it is possible to relax positioning accuracy in stacking and bonding the respective layer substrates and contribute to simplification of the manufacturing process.

### [Modifications]

The configuration of the image pickup apparatus (the image pickup module) of the present invention is not limited to the configuration example in the embodiment explained above and can also be further configured as a different form. Various modifications about the image pickup apparatus (the image pickup module) in the present embodiment are explained below.

FIG. 11 is a diagram showing a first modification of the image pickup apparatus (the image pickup module) in the embodiment of the present invention. FIG. 11 is a sectional view of a part equivalent to the cross section taken along the surface indicated by the arrow sign [3] in FIG. 2 in the image pickup apparatus in the embodiment explained above.

As shown in FIG. 11, a basic configuration in the first modification of the present embodiment includes substantially the same components as the components in the embodiment explained above. The present modification is different only in a configuration of a first layer substrate 11A of a first member 8A. Therefore, in explanation of the present modification, only components different from the components in the embodiment explained above are explained in detail. In the configuration in the present modification (FIG. 11), the same components as the components in the embodiment explained above (see FIG. 3) are denoted by the same reference numerals and signs and explanation of the components is omitted.

In other words, in an image pickup module 10A in the first modification, the first layer substrate 11A configuring a part of the first member 8A is formed as a whole by a member (for example, a transparent resin material) that transmits light. With this configuration, in the image pickup module 10A in the first modification, the first layer substrate 11A itself functions as the illumination window 15 and the observation window 16 in the embodiment explained above. The other components are the same as the components in the embodiment explained above.

According to the first modification configured as explained above, since the first layer substrate 11A directly has the functions of the illumination window 15 and the observation window 16, it is possible to omit a step of forming an opening in the first layer substrate, a step of two-color molding for a transparent window member, and the like. Therefore, it is possible to contribute to simplification of a manufacturing process and more easily manufacture the image pickup module 10A. It is also possible to contribute to a reduction in manufacturing cost by simplifying the manufacturing process.

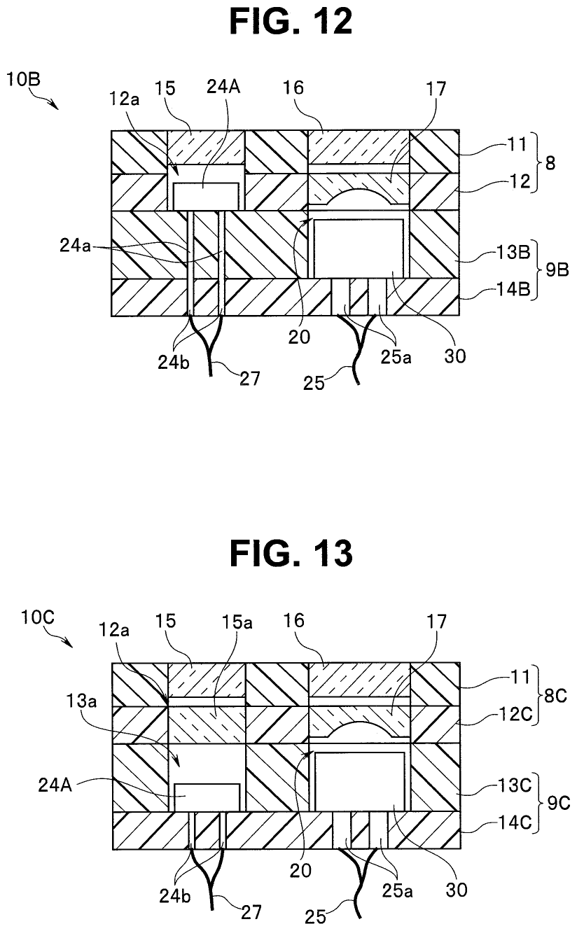

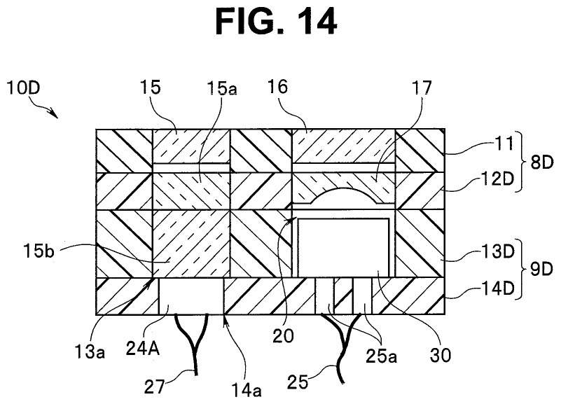

FIG. 12 to FIG. 14 are sectional views respectively showing three configuration examples in a second modification of the image pickup apparatus (the image pickup module) in the embodiment of the present invention. Among the figures, FIG. 12 is a sectional view showing a first configuration example in the second modification. FIG. 13 is a sectional view showing a second configuration example in the second modification. FIG. 14 is a sectional view showing a third configuration example in the second modification.

FIG. 12 to FIG. 14 are, like FIG. 11 (the first modification), sectional views of the part equivalent to the cross section taken along the surface indicated by the arrow sign [3] in FIG. 2 in the image pickup apparatus in the embodiment explained above.

In the first to third configuration examples in the second modification, a basic configuration includes substantially the same components as the components in the embodiment explained above. Therefore, in explanation of the respective configuration examples in the second modification as well, only components different from the components in the embodiment explained above are explained in detail. In the respective configuration examples (FIG. 12 to FIG. 14) in the second modification, the same components as the components (see FIG. 3) in the embodiment explained above are denoted by the same reference numerals and signs and explanation of the components is omitted.

In the respective configuration examples (FIG. 12 to FIG. 14) in the second modification, a configuration of an illumination unit is different from the configuration in the embodiment explained above.

The illumination unit illustrated in the embodiment explained above is configured by the illumination window 15, the illumination optical path 19, and the illumination member 24 as shown in FIG. 3. In this case, the light guide cable or the like is applied as the illumination member 24.

As opposed to such a configuration in the embodiment, in the respective configuration examples (FIG. 12 to FIG. 14) in the second modification, a case is illustrated in which a light emitting element 24A such as a light emitting diode (LED) is applied as an illumination member of an illumination unit.

First, as shown in FIG. 12, in an image pickup module 10B in the first configuration example in the second modification, a configuration is illustrated in which the light emitting element 24A is disposed in the illumination opening 12a formed in the second layer substrate 12 of the first member 8. In this case, the light emitting element 24A is the first illumination element. According to this configuration, illumination wires 24a and 24b are provided on a third layer substrate 13B and a fourth layer substrate 14B of a second member 9B instead of the illumination openings 13a and 14a (the second and third illumination elements) in the embodiment explained above. In the first configuration example in the second modification, the illumination wires 24a and 24b are the second and third illumination elements.

Here, like the image pickup signal wires 25a, the illumination wires 24a and 24b are Si through-electrodes (TSVs) or the like. Therefore, the illumination wires 24a and 24b can be formed by the same method as the method of forming the image pickup signal wires 25a.

One end of the illumination wire 24a is connected to the light emitting element 24A and the other end is connected to the illumination wire 24b. One end of the illumination wire 24b is connected to the illumination wire 24a and the other end is connected to an electric cable 27. Note that the electric cable 27 is a power line that is inserted through an insertion section, an operation section, a universal cord, and a connector of an endoscope and transmits, to the light emitting element 24A, electric power supplied from external peripheral equipment (not shown) to which the connector is connected.

Note that the illumination wires 24a and 24b may be separately formed and electrically connected to each other as explained above or the illumination wires 24a and 24b may be integrally formed. The other components are the same as the components in the embodiment explained above.

Subsequently, as shown in FIG. 13, a configuration is illustrated in which, in an image pickup module 10C in the second configuration example in the second modification, the light emitting element 24A is disposed in the illumination opening 13a formed in a third layer substrate 13C of a second member 9C. In this case, the light emitting element 24A is the second illumination element. According to this configuration, the illumination wire 24b is provided on a fourth layer substrate 14C of the second member 9C instead of the illumination opening 14a in the embodiment explained above. In the second configuration example in the second modification, the illumination wire 24b is the third illumination element. Note that the light emitting element 24A is connected to one end of the illumination wire 24b and the electric cable 27 is connected to the other end.

In the illumination opening 12a formed in a second layer substrate 12C of a first member 8C, for example, an illumination lens 15a, which is an optical member formed by a concave lens or the like, is disposed. The illumination lens 15a forms a part of an illumination optical system having a function of diffusing illumination light emitted from the light emitting element 24A. In this case, the illumination lens 15a is the first illumination element. Note that, like the observation lens 17, the illumination lens 15a can be molded by molding in the second layer substrate 12C.

As explained above, by integrally providing the illumination lens 15a in the second layer substrate 12C, the illumination unit can efficiently radiate the illumination light emitted from the light emitting element 24A toward the examination target site in the front through the illumination window 15.

Note that, in the illumination unit, the illumination light emitted from the light emitting element 24A only has to be able to be radiated toward the examination target site in the front through the illumination window 15. Therefore, instead of providing the illumination lens 15a, as in the embodiment explained above, the illumination opening 12a functioning as a mere through-hole may be molded as the first illumination element.

Meanwhile, as shown in FIG. 14, in an image pickup module 10D in the third configuration example in the second modification, a configuration is shown in which the light emitting element 24A is disposed in the illumination opening 14a formed in a fourth layer substrate 14D of a second member 9D. In this case, the light emitting element 24A is the third illumination element. The electric cable 27 is connected to the light emitting element 24A.

In the illumination opening 12a formed in the second layer substrate 12C of the first member 8C, the illumination lens 15a having the same configuration as the third configuration example (see FIG. 13) explained above is disposed. The illumination lens 15a is the first illumination element molded by, for example, molding integrally with the second layer substrate 12C.

Further, in the third configuration example in the second modification, in the illumination opening 13a formed in a third layer substrate 13D of the second member 9D, a second illumination lens 15b that is the same as the illumination lens 15a explained above is disposed. The second illumination lens 15b is the second illumination element molded by, for example, molding integrally with the third layer substrate 13D.

Note that, in an illumination unit, illumination light emitted from the light emitting element 24A only has to be able to be radiated toward the examination target site in the front through the illumination window 15. Therefore, instead of providing the illumination lens 15a and the second illumination lens 15b, as in the embodiment and the like explained above, the illumination openings 12a and 13a functioning as mere through-holes may be molded as the first and second illumination elements or may be configured by combining lenses and openings as appropriate.

With the respective configuration examples in the second modification explained above, since the light emitting element 24A is applied as the illumination member of the illumination unit, the illumination unit including the illumination member (the light source) can be packaged at the distal end rigid portion of the distal end portion of the endoscope. Therefore, it is possible to simplify an overall configuration compared with a configuration in which a light guide cable is drawn around. At the same time, it is possible to contribute to a reduction in a manufacturing process and more easily manufacture an image pickup module. Furthermore, it is possible to contribute to a reduction in manufacturing cost as well according to the reduction in the manufacturing process.

In the respective configuration examples in the second modification explained above, a configuration is illustrated in which the light emitting element 24A is disposed on the second to fourth layer substrates. In order to realize the configuration, for example, as illustrated above, there is a form in which the light emitting element 24A is mounted on the rigid substrate. As a different example, for example, a compound semiconductor substrate or the like is used as the rigid substrate forming the respective layer substrates. Consequently, the light emitting element 24A can be directly formed in the substrate.

The endoscope of the present invention can be naturally applied to an endoscope for medical use (for a living body) that uses a living body as a subject and is inserted into the living body to perform observation and the like of an examination target site in the living body. Besides, the endoscope of the present invention can also be applied to, completely in the same manner, an endoscope for industrial use inserted into, for example, a pipe such as a water service pipe and used for, for example, observation of an inside of the pipe.

The present invention is not limited to the embodiment explained above. It goes without saying that various modifications and applications can be implemented within a range not departing from the gist of the invention. Further, inventions in various stages are included in the embodiment. Various inventions can be extracted by appropriate combinations in a disclosed plurality of constituent elements. For example, when the problems to be solved by the invention can be solved and the effects of the invention can be obtained even if several constituent elements are deleted from all the constituent elements described in the embodiment, configurations in which the constituent elements are deleted can be extracted as inventions. Further, constituent elements described in different embodiments may be combined as appropriate. The present invention is not restricted by specific implementation modes of the invention except that the invention is limited by the appended claims.

What is claimed is:

1. An image pickup apparatus comprising:
a first member including an illumination optical element and an observation optical element respectively formed by members that transmit light;
an image pickup device; and
a second member including a signal wire electrically connected to the image pickup device and an illumination member disposed in a position corresponding to the illumination optical element, wherein
the first member includes a first layer substrate including an illumination window and an observation window respectively formed by members that transmit light and a second layer substrate including a first illumination element and an observation lens,
the second member includes a third layer substrate including a second illumination element and an opening in which the image pickup device is disposed and a fourth layer substrate including a third illumination element and a signal wire electrically connected to the image pickup device,
the first member and the second member are configured in a form in which the first layer substrate, the second layer substrate, the third layer substrate, and the fourth layer substrate are stacked in this order and integrally bonded, and
the first illumination element is a light emitting element.

2. The image pickup apparatus according to claim 1, wherein
an illumination apparatus is formed by the illumination window, the first illumination element, the second illumination element, the third illumination element, and the illumination member,
an observation apparatus is formed by the observation window, the observation lens, and the image pickup device, and
each of the illumination window, the first illumination element, the second illumination element, the third illumination element, the observation window, the observation lens, and the image pickup device is positioned in a plane orthogonal to a stacking direction of the first layer substrate, the second layer substrate, the third layer substrate, and the fourth layer substrate.

3. The image pickup apparatus according to claim 2, wherein the observation optical system is configured such that an optical axis of the observation lens and a center point of a light receiving surface of the image pickup device coincide.

4. The image pickup apparatus according to claim 2, wherein, in the observation optical system, the observation window, the observation lens, and the image pickup device are disposed to be arrayed in a row in the stacking direction.

5. The image pickup apparatus according to claim 1, wherein the first member includes:
a first rigid substrate;
the illumination window formed integrally with the first rigid substrate; and
the observation window formed integrally with the first rigid substrate.

6. The image pickup apparatus according to claim 1, wherein the first member is entirely formed by a member that transmits light.

7. The image pickup apparatus according to claim 1, wherein the second illumination element is a second opening piercing through a part of the second member in a stacking direction of the second member.

8. The image pickup apparatus according to claim 7, wherein the third illumination element is a third opening piercing through a part of the second member in the stacking direction of the second member.

9. The image pickup apparatus according to claim 7, wherein the third illumination element is a light emitting element.

10. The image pickup apparatus according to claim 1, wherein thickness in a stacking direction of the third layer substrate is defined by a focal length of the observation lens and a thickness dimension of the image pickup device.

11. The image pickup apparatus according to claim 8, wherein, when the first member and the second member are stacked, the first opening, the second opening, and the third opening communicate to form a first through-hole.

12. An image pickup apparatus comprising:
a first member including an illumination optical element and an observation optical element respectively formed by members that transmit light;
an image pickup device; and
a second member including a signal wire electrically connected to the image pickup device and an illumination member disposed in a position corresponding to the illumination optical element, wherein
the first member includes a first layer substrate including an illumination window and an observation window respectively formed by members that transmit light and a second layer substrate including a first illumination element and an observation lens,
the second member includes a third layer substrate including a second illumination element and an opening in which the image pickup device is disposed and a fourth layer substrate including a third illumination element and a signal wire electrically connected to the image pickup device,
the first member and the second member are configured in a form in which the first layer substrate, the second layer substrate, the third layer substrate, and the fourth layer substrate are stacked in this order and integrally bonded,
the first illumination element is a first opening piercing through a part of the first member in a stacking direction of the first member, and
the second illumination element is a light emitting element.

13. An image pickup apparatus comprising:
a first member including an illumination optical element and an observation optical element respectively formed by members that transmit light;
an image pickup device; and
a second member including a signal wire electrically connected to the image pickup device and an illumination member disposed in a position corresponding to the illumination optical element, wherein
the first member includes a first layer substrate including an illumination window and an observation window respectively formed by members that transmit light and a second layer substrate including a first illumination element and an observation lens,
the second member includes a third layer substrate including a second illumination element and an opening in which the image pickup device is disposed and a fourth layer substrate including a third illumination element and a signal wire electrically connected to the image pickup device,
the first member and the second member are configured in a form in which the first layer substrate, the second layer substrate, the third layer substrate, and the fourth layer substrate are stacked in this order and integrally bonded,
each of the first layer substrate, the second layer substrate, the third layer substrate, and the fourth layer substrate includes two openings piercing through the layer substrate in a stacking direction of the layer substrate, and
when the first layer substrate, the second layer substrate, the third layer substrate, and the fourth layer substrate are stacked, the respective openings communicate to form two through-holes.

14. An endoscope comprising an image pickup apparatus, the image pickup apparatus including:
a first member including an illumination optical element and an observation optical element respectively formed by members that transmit light;
an image pickup device; and
a second member including a signal wire electrically connected to the image pickup device and an illumination member disposed in a position corresponding to the illumination optical element, wherein
the first member includes a first layer substrate including an illumination window and an observation window respectively formed by members that transmit light and a second layer substrate including a first illumination element and an observation lens,
the second member includes a third layer substrate including a second illumination element and an opening in which the image pickup device is disposed and a fourth layer substrate including a third illumination element and a signal wire electrically connected to the image pickup device,
the first member and the second member are configured in a form in which the first layer substrate, the second layer substrate, the third layer substrate, and the fourth layer substrate are stacked in this order and integrally bonded, and
the first illumination element is a light emitting element.

15. A manufacturing method for an image pickup apparatus comprising:
manufacturing a first layer substrate including an illumination window and an observation window respectively formed by members that transmit light and an insertion opening for a channel pipe;
manufacturing a second layer substrate including a first illumination element, an observation lens, and an insertion opening for the channel pipe;
manufacturing a fourth layer substrate including a third illumination element, a signal wire, and an insertion opening for the channel pipe;
connecting an image pickup device to the signal wire and mounting the image pickup device on the fourth layer substrate;
manufacturing, on the fourth layer substrate, a third layer substrate including a second illumination element, an opening in which the image pickup device is disposed, and an insertion opening for the channel pipe; and
stacking the first layer substrate, the second layer substrate, the third layer substrate, and the fourth layer substrate in order and bonding the respective layer substrates in a state in which the respective insertion openings communicate and a through-hole is formed.
